(19) United States
(12) Patent Application Publication
(10) Pub. No.: US 2024/0408128 A1
(43) Pub. Date: Dec. 12, 2024

(54) TOPICAL PHARMACEUTICAL COMPOSITIONS FOR TREATMENT OF INFERTILITY

(71) Applicant: CHEMEX GLOBAL, Anand, Gujarat (IN)

(72) Inventor: Nirav JADAV, Anand, Gujarat (IN)

(73) Assignee: CHEMEX GLOBAL, Anand, Gujarat (IN)

(21) Appl. No.: 18/691,629

(22) PCT Filed: Sep. 13, 2022
(86) PCT No.: PCT/IN2022/050817

§ 371 Date: Mar. 13, 2024

(30) Foreign Application Priority Data
Sep. 15, 2021 (IN) 202121041542

(51) Int. Cl. A61K 31/728; A61K 9/00; A61K 9/06; A61K 31/198; A61K 31/4166; A61K 36/185; A61K 36/258; A61K 36/886; A61K 47/02; A61K 47/10; A61K 47/26; A61K 47/36

(52) U.S. Cl. CPC A61K 31/728; A61K 9/0014; A61K 9/06; A61K 31/198; A61K 31/4166; A61K 36/185; A61K 36/258; A61K 36/886; A61K 47/02; A61K 47/10; A61K 47/26; A61K 47/36

(57) ABSTRACT

The invention relates to vaginal pharmaceutical compositions containing fertility aid or pharmaceutically acceptable salts thereof, for use as adjuvant therapy or supportive aid in the treatment of infertility. Further, the present invention provides a process for the preparation of the said composition.

### FIELD OF INVENTION

The invention relates to vaginal pharmaceutical compositions containing fertility aid or pharmaceutically acceptable salts thereof, for use as adjuvant therapy or supportive aid in the treatment of infertility. Further, the present invention provides a process for the preparation of the said composition.

### BACKGROUND OF THE INVENTION

Infertility and subfertility affect a significant proportion of humanity.

Infertility is a medical condition that can cause psychological, physical, mental, spiritual, and medical detriments to the patient. The unique quality of this medical condition involves affecting both the patient and the patient's partner as a couple.

Infertility is a disease in which the ability to get pregnant and give birth to a child is impaired or limited in some way. When the cause of the infertility is found to come from the female partner, it is considered female infertility or “female factor” infertility. This is typically diagnosed after a woman has tried to get pregnant (through unprotected sex) for 12 months or longer without a pregnancy. However, there is no specified time period for conclusion of female infertility.

The World Health Organization (WHO) performed a large multinational study to determine gender distribution and infertility etiologies. In 37% of infertile couples, female infertility was the cause; in 35% of couples, both male and female causes were identified; in 8%, there was male factor infertility. Infertility is a common problem of about 6% of women aged 15 to 44.

Pregnancy is the result of a process that has many steps.

To get pregnant 1) A woman's body must release an egg from one of her ovaries, 2) A man's sperm must join with the egg along the way (fertilize), 3) The fertilized egg must go through a fallopian tube toward the uterus (womb), 4) The embryo must attach to the inside of the uterus (implantation), 5) Infertility may result from a problem with any or several of these steps.

Broadly, Infertility can be due to identified causes and unidentified causes. These identified causes and an unidentified cause mainly affects both women and men.

Some possible causes of female factor of identified infertility can include:
Problems with the uterus: This includes polyps, fibroids, septum or adhesions inside the cavity of the uterus. Polyps and fibroids can form on their own at any time, whereas other abnormalities (like a septum) are present at birth. Adhesions can form after a surgery like a dilation and curettage (D&C).
Problems with the fallopian tubes: The most common cause of “tubal factor” infertility is pelvic inflammatory disease, usually caused by chlamydia and gonorrhea.
Problems with ovulation: There are many reasons why a woman may not ovulate (release an egg) regularly. Hormonal imbalances, a past eating disorder, substance abuse, thyroid conditions, severe stress and pituitary tumors are all examples of things that can affect ovulation.
Problems with egg number and quality: Women are born with all the eggs they will ever have, and this supply can “run out” early before menopause. In addition, some eggs will have the wrong number of chromosomes and cannot fertilize or grow into a healthy fetus. Some of these chromosomal issues (such as “balanced translocation”) may affect all of the eggs. Others are random but become more common as a woman gets older.

There are many treatment options for identified infertility, including medications to correct hormonal issues, surgery for physical problems and in vitro fertilization (IVF).

Some common medicines used to treat identified infertility in women include:
Clomiphene citrate (Clomid®*) is a medicine that causes ovulation by acting on the pituitary gland. It is often used in women who have polycystic ovary syndrome (PCOS) external icon or other problems with ovulation. It is also used in women with normal ovulation to increase the number of mature eggs produced. This medicine is taken by mouth.
Letrozole (Femara®*) is a medication that is frequently used off-label to cause ovulation. It works by temporarily lowering a woman's progesterone level, which causes the brain to naturally make more FSH. It is often used to induce ovulation in woman with PCOS, and in women with normal ovulation to increase the number of mature eggs produced in the ovaries. It is taken by mouth.
Human menopausal gonadotropin or hMG (Menopur®*; Repronex®*; Pergonal®*) is an injectable medication often used for women who don't ovulate because of problems with their pituitary gland—hMG acts directly on the ovaries to stimulate development of mature eggs.
Follicle-stimulating hormone or FSH (Gonal-F®*; Follistim®*) is an injectable medication that works much like hMG. It stimulates development of mature eggs within the ovaries.
Gonadotropin-releasing hormone (GnRH) analogs and GnRH antagonists are medications that act on the pituitary gland to prevent a woman from ovulating. They are used during in vitro fertilization cycles, or to help prepare a woman's uterus for an embryo transfer. These medications are usually injected or given with a nasal spray.
Metformin (Glucophage®*) is a medicine doctors use for women who have insulin resistance or diabetes and PCOSexternal icon. This drug helps lower the high levels of male hormones in women with these conditions. This helps the body to ovulate. Sometimes clomiphene citrate or FSH is combined with metformin. This medicine is taken by mouth.
Bromocriptine (Parlodel®*) and Cabergoline (Dostinex®*) are medications used for women with ovulation problems because of high levels of prolactin. These medications are taken by mouth.

Impaired fecundity is a condition related to infertility and refers to women who have difficulty getting pregnant or carrying a pregnancy to term.

Assisted Reproductive Technology (ART) includes all fertility treatments in which either eggs or embryos are handled outside of the body. In general, ART procedures involve removing mature eggs from a woman's ovaries using a needle, combining the eggs with sperm in the laboratory, and returning the embryos to the woman's body or donating them to another woman. Below are the different types of assisted reproductive technology (ART):
Intracytoplasmic sperm injection (ICSI) is a type of IVF that is often used for couples with male factor infertility. With ICSI, a single sperm is injected into a mature egg. The alternative to ICSI is “conventional” fertilization where the egg and many sperm are placed in a petri dish together and the sperm fertilizes an egg on its own.
In vitro fertilization (IVF), meaning fertilization outside of the body, is the most common form of ART. Eggs and sperm are combined in a laboratory to create embryos. After about three to five days, the embryo (or embryos) is transferred into the woman's uterus. Embryos can also be frozen for a future transfer. When a frozen embryo is thawed and transferred into a woman's uterus it is called a frozen embryo transfer (FET).

Women who have poor egg quality, are older, or who have not had success with previous IVF cycles, may choose to consider IVF with donor eggs and her partner's sperm. The resulting baby is biologically related to the father and not the mother, although the mother carries the pregnancy. IVF using fresh embryos from donor eggs has a high success rate, resulting in live births 55% of the time.

Blastocyst transfer is a relatively new IVF technology. Traditionally, IVF embryos were transferred to the uterus when they were at a stage of having 2 to 8 cells. In this procedure, the embryos grow for five days until they reach a later stage of development known as the blastocyst stage. Then, one or two blastocysts are transferred to the uterus. This eliminates the possibility of triplets and retains the high success rate of IVF.

Donor embryos are embryos donated by couples who have completed the IVF process. Transferring donor embryos is less costly than standard IVF or IVF with donor eggs. This procedure allows the experience of pregnancy. The baby will be biologically unrelated to either parent.

Zygote intrafallopian transfer (ZIFT) or tubal embryo transfer and gamete intrafallopian transfer (GIFT) are other ART methods that are rarely used in the United States today. With ZIFT, fertilization occurs in the laboratory similar to IVF. Then the very young embryo is transferred to the fallopian tube instead of the uterus. GIFT involves transferring eggs and sperm into the woman's fallopian tube and fertilization occurs in the woman's body.

Preimplantation genetic testing is a procedure used to identify genetic disorders or chromosomal abnormalities in embryos created during an IVF cycle. One or more cells are biopsied from each embryo and sent for testing.

Surrogacy can be an option for women who have trouble carrying a pregnancy to term. Traditional surrogacy involves insemination of the surrogate with the male partner's sperm. Gestational surrogacy is another option that involves using IVF to create embryos from both partners and transferring these embryos to the uterus of the surrogate. This option allows the baby to be biologically related to both the male and female partners.

Fertility can be naturally busted by few lifestyle changes and can increase chances of a healthy pregnancy.

Some possible causes of male factor of identified infertility can include:
Varicocele. A varicocele is a swelling of the veins that drain the testicle. It's the most common reversible cause of male infertility. Although the exact reason that varicoceles cause infertility is unknown, it may be related to abnormal blood flow. Varicoceles lead to reduced sperm quantity and quality.
Infection. Some infections can interfere with sperm production or sperm health or can cause scarring that blocks the passage of sperm. These include inflammation of the epididymis (epididymitis) or testicles (orchitis) and some sexually transmitted infections, including gonorrhea or HIV. Although some infections can result in permanent testicular damage, most often sperm can still be retrieved.
Ejaculation issues. Retrograde ejaculation occurs when semen enters the bladder during orgasm instead of emerging out the tip of the penis. Various health conditions can cause retrograde ejaculation, including diabetes, spinal injuries, medications, and surgery of the bladder, prostate or urethra.
Antibodies that attack sperm. Anti-sperm antibodies are immune system cells that mistakenly identify sperm as harmful invaders and attempt to eliminate them.
Tumors. Cancers and nonmalignant tumors can affect the male reproductive organs directly, through the glands that release hormones related to reproduction, such as the pituitary gland, or through unknown causes. In some cases, surgery, radiation or chemotherapy to treat tumors can affect male fertility.
Undescended testicles. In some males, during fetal development one or both testicles fail to descend from the abdomen into the sac that normally contains the testicles (scrotum). Decreased fertility is more likely in men who have had this condition.
Hormone imbalances. Infertility can result from disorders of the testicles themselves or an abnormality affecting other hormonal systems including the hypothalamus, pituitary, thyroid and adrenal glands. Low testosterone (male hypogonadism) and other hormonal problems have a number of possible underlying causes.
Defects of tubules that transport sperm. Many different tubes carry sperm. They can be blocked due to various causes, including inadvertent injury from surgery, prior infections, trauma or abnormal development, such as with cystic fibrosis or similar inherited conditions.

Blockage can occur at any level, including within the testicle, in the tubes that drain the testicle, in the epididymis, in the vas deferens, near the ejaculatory ducts or in the urethra.
Chromosome defects. Inherited disorders such as Klinefelter's syndrome-in which a male is born with two X chromosomes and one Y chromosome (instead of one X and one Y)-cause abnormal development of the male reproductive organs. Other genetic syndromes associated with infertility include cystic fibrosis and Kallmann's syndrome.
Problems with sexual intercourse. These can include trouble keeping or maintaining an erection sufficient for sex (erectile dysfunction), premature ejaculation, painful intercourse, anatomical abnormalities such as having a urethral opening beneath the penis (hypospadias), or psychological or relationship problems that interfere with sex.
Celiac disease. Celiac disease is a digestive disorder caused by sensitivity to a protein found in wheat called gluten. The condition may contribute to male infertility. Fertility may improve after adopting a gluten-free diet.
Certain medications. Testosterone replacement therapy, long-term anabolic steroid use, cancer medications (chemotherapy), some ulcer drugs, some arthritis drugs and certain other medications can impair sperm production and decrease male fertility.
Prior surgeries. Certain surgeries may prevent you from having sperm in your ejaculate, including vasectomy, scrotal or testicular surgeries, prostate surgeries, and large abdominal surgeries performed for testicular and rectal cancers, among others.
Industrial chemicals. Extended exposure to certain chemicals, pesticides, herbicides, organic solvents and painting materials may contribute to low sperm counts.
Heavy metal exposure. Exposure to lead or other heavy metals also may cause infertility.
Radiation or X-rays. Exposure to radiation can reduce sperm production, though it will often eventually return to normal. With high doses of radiation, sperm production can be permanently reduced.
Overheating the testicles. Elevated temperatures may impair sperm production and function. Although studies are limited and are inconclusive, frequent use of saunas or hot tubs may temporarily impair your sperm count.
Drug use. Anabolic steroids taken to stimulate muscle strength and growth can cause the testicles to shrink and sperm production to decrease. Use of cocaine or marijuana may temporarily reduce the number and quality of your sperm as well.
Alcohol use. Drinking alcohol can lower testosterone levels, cause erectile dysfunction and decrease sperm production. Liver disease caused by excessive drinking also may lead to fertility problems.
Tobacco smoking. Men who smoke may have a lower sperm count than do those who don't smoke. Secondhand smoke also may affect male fertility.
Weight. Obesity can impair fertility in several ways, including directly impacting sperm themselves as well as by causing hormone changes that reduce male fertility.

Some common treatment used to treat identified infertility in men includes:
Surgery. For example, a varicocele can often be surgically corrected or an obstructed vas deferens repaired. Prior vasectomies can be reversed. In cases where no sperm are present in the ejaculate, sperm can often be retrieved directly from the testicles or epididymis using sperm retrieval techniques.
Treating infections. Antibiotic treatment might cure an infection of the reproductive tract, but doesn't always restore fertility.
Treatments for sexual intercourse problems. Medication or counseling can help improve fertility in conditions such as erectile dysfunction or premature ejaculation.
Hormone treatments and medications. Your doctor might recommend hormone replacement or medications in cases where infertility is caused by high or low levels of certain hormones or problems with the way the body uses hormones.
Assisted reproductive technology (ART). ART treatments involve obtaining sperm through normal ejaculation, surgical extraction or from donor individuals, depending on your specific case and wishes. The sperm are then inserted into the female genital tract, or used to perform in vitro fertilization or intracytoplasmic sperm injection.

There are unidentified condition for infertility in men and women like, Modern habits (smoking, Alcohol, chemical exposure, Junk food etc), Changing environment, Personal and professional Stress, Modern lifestyle, Late age marriages, Tendency to family plan once settle financially, Imbalance family life, Technology radiation, increase mobile usage, lack of exercise, Both person working culture, Improper genial hygiene etc.

Despite all this available options Trying to Conceive (TCC) couple have tremendous sociocultural stress, family pressure, and fear of image damage in society.

There are very less treatment option available for unidentified infertility. TTC will to any extend to resolve this problem without worrying for treatment cost. IVF and other techniques are not sure shot treatment and highly costly. There is no concrete affordable Fertility aid.

There are few products available in market like Conceive Plus®, Pre-seed®, KY Jelly®, Fertility Gel®, Sure Babi®. However, these are costly. Further, there is still need for the development of Fertility aid composition which maintain health of sperm. Hence, there is need for the development of cost effective composition which utilized as adjuvant therapy or supportive aid in the treatment of identified and unidentified cause of infertility.

Further, one of the cause of infertility is insufficient amount of cervical mucus of vagina in women. Sperm cannot survive in environment where less amount of cervical mucus is present. This ultimately results in infertility. Further, insufficient amount of cervical mucus results in painful intercourse. Hence, there is also need to develop composition which prevent vaginal dryness by providing environment to semen which resembles to human cervical mucus and provide smooth intercourse.

Pharmaceutical composition of present invention provides topical formulation which is utilized as adjuvant therapy or supportive aid in the treatment of identified and unidentified cause of infertility in men and women.

### OBJECT OF THE INVENTION

The main object of the present invention is to provide a stable topical pharmaceutical composition comprising fertility aids or pharmaceutically acceptable salts thereof.

Another object of the present invention is to provide a stable topical pharmaceutical composition comprising fertility aids or pharmaceutically acceptable salts thereof, wherein the said composition is a gel, cream, an ointment or a lotion.

Another object of the present invention is to provide a stable topical pharmaceutical composition comprising fertility aids or pharmaceutically acceptable salts thereof, and at least one pharmaceutically acceptable excipient, wherein the pH of the said composition is more than 5.

Another object of the present invention is to provide a stable topical pharmaceutical composition comprising fertility aids or pharmaceutically acceptable salts thereof and at least one pharmaceutically acceptable excipient, wherein the pH of the said composition is from 5 to 8.

Another object of the present invention is to provide a stable gel or solution or spray for solution comprising fertility aids or pharmaceutically acceptable salts thereof, and at least one pharmaceutically acceptable excipient selected from a gelling agent, soothing agent, preservative, antioxidant, wetting agent, solubilizers, humectant, buffering agent, surfactants, moisturizing agent, emollient, chelating agents, solvents or mixture thereof.

Another object of the present invention is to provide a process for preparation of stable topical pharmaceutical composition comprising fertility aid or pharmaceutically acceptable salts thereof, wherein the said composition is a solution or a solution for spray.

Another object of the present invention is to provide a process for preparation of stable topical pharmaceutical composition comprising fertility aid or pharmaceutically acceptable salts thereof, wherein the said composition is a gel prepared by mixing the components in the water phase.

Another object of the present invention is to provide a stable topical pharmaceutical composition comprising fertility aid or pharmaceutically acceptable salts thereof, wherein the said composition is stable when stored at 25° C./60% RH for at least 12 months.

Another object of the present invention is to provide a stable topical pharmaceutical composition comprising fertility aid or pharmaceutically acceptable salts thereof, wherein the said composition is stable when stored at 40° C./75% RH for at least 6 months.

Another object of the present invention refers to single layer or multilayer laminated aluminum collapsible tube, spray pump, single use cartridge seamless plastic tube, elongated nozzle tube tube with suitable applicator packaging container comprising a stable topical gel comprising fertility aid or pharmaceutically acceptable salts thereof.

Another object of the present invention refers to single layer or multilayer laminated aluminum collapsible tube, spray pump, single use cartridge seamless plastic tube, elongated nozzle tube tube with suitable applicator packaging container comprising a stable topical solution or solution for spray comprising fertility aid or pharmaceutically acceptable salts thereof.

Another object of the present invention refers to single layer or multilayer laminate packaging container comprising a stable topical solution or solution for spray comprising fertility aid or pharmaceutically acceptable salts thereof.

Another object of the present invention relates to a stable topical pharmaceutical composition comprising fertility aid or pharmaceutically acceptable salts thereof, wherein the said composition can be used for female or male administration for adjuvant therapy or supportive aid of infertility, wherein the said infertility is identified or unidentified.

Another object of the present invention relates to a stable topical pharmaceutical composition comprising fertility aid or pharmaceutically acceptable salts thereof, wherein the said composition can be used to avoid vaginal dryness and thereby prevent painful intercourse.

### SUMMARY OF THE INVENTION

In a first embodiment, the present invention relates to a stable topical pharmaceutical composition comprising fertility aid or pharmaceutically acceptable salts thereof.

In another embodiment, the present invention relates to a stable topical pharmaceutical composition comprising fertility aid or pharmaceutically acceptable salts thereof, wherein the said composition is a gel, cream, an ointment or a lotion.

In another embodiment, the present invention relates to a stable topical pharmaceutical composition comprising fertility aid or pharmaceutically acceptable salts thereof, wherein the said composition is solution, or solution for spray.

In another embodiment, the present invention relates to a stable topical pharmaceutical composition comprising fertility aid or pharmaceutically acceptable salts thereof with at least one pharmaceutically acceptable excipient, wherein the pH of the said composition is more than 5.

In another embodiment, the present invention relates to a stable topical pharmaceutical composition comprising fertility aid or pharmaceutically acceptable salts thereof and at least one pharmaceutically acceptable excipient, wherein the pH of the said composition is from 5 to 8.

In another embodiment, the present invention relates to a stable topical pharmaceutical composition comprising fertility aid or pharmaceutically acceptable salts thereof and at least one pharmaceutically acceptable excipient, wherein the pH of the said composition is from 5 to 8 and the said pH do not harm the semen or sperm.

In another embodiment, the present invention relates to a stable topical pharmaceutical composition comprising fertility aid or pharmaceutically acceptable salts thereof, and pharmaceutically acceptable excipients selected from gelling agent, soothing agent, preservative, antioxidant, wetting agent, solubilizers, humectant, buffering agent, surfactants, moisturizing agent, emollients, chelating agents, solvents or mixture thereof.

In another embodiment, the present invention relates to a process for preparation of stable topical pharmaceutical composition comprising fertility aid or pharmaceutically acceptable salts thereof, wherein the said composition is a gel prepared by mixing the components in the water phase.

In another embodiment, the present invention relates to a process for preparation of stable topical pharmaceutical composition comprising fertility aid or pharmaceutically acceptable salts thereof, wherein the said composition is a solution or solution for spray is prepared by mixing the components in the water phase at proper temperature.

In another embodiment, the present invention relates to a stable topical pharmaceutical composition comprising fertility aid or pharmaceutically acceptable salts thereof, wherein the said composition is stable when stored at 25° C./60% RH for at least 12 months.

In another embodiment, the present invention relates to a stable topical pharmaceutical composition comprising fertility aid or pharmaceutically acceptable salts thereof, wherein the said composition is stable when stored at 40° C./75% RH for at least 6 months.

In another embodiment, the present invention relates to a stable topical pharmaceutical composition comprising fertility aid or pharmaceutically acceptable salts thereof, wherein the said fertility aid or pharmaceutically acceptable salts thereof does not penetrate into systemic circulation, wherein the said fertility aid or pharmaceutically acceptable salts thereof penetrate into systemic circulation in non-detectable amount.

In another embodiment, the present invention relates to a stable topical pharmaceutical composition comprising fertility aid or pharmaceutically acceptable salts thereof, wherein the said topical composition provides irritation free effect.

In another embodiment, the present invention relates to a stable topical pharmaceutical composition comprising fertility aid or pharmaceutically acceptable salts thereof, wherein the said composition can be used for female or male administration for adjuvant therapy or supportive aid of infertility, wherein the said infertility is identified or unidentified.

In another embodiment, the present invention relates to a stable topical pharmaceutical composition comprising fertility aid or pharmaceutically acceptable salts thereof, wherein the said composition can be used by trying to conceive female or male for adjuvant therapy or supportive aid of infertility, wherein the said infertility is identified or unidentified.

In another embodiment, the present invention relates to a stable topical pharmaceutical composition comprising fertility aid or pharmaceutically acceptable salts thereof, wherein the said composition can be used for trying to conceive female or male for adjuvant therapy or supportive aid of infertility, wherein the said composition is use separately or simultaneously in the treatment of infertility.

In another embodiment, the present invention relates to a stable topical pharmaceutical composition comprising fertility aid or pharmaceutically acceptable salts thereof, wherein the said composition can be used to avoid vaginal dryness and thereby prevent painful intercourse.

### DETAILED DESCRIPTION

The detailed description and the examples provided herein are exemplary and any modification or variation within the scope of the invention will be apparent to a person skilled in the art. Further, unless otherwise defined, all the technical and scientific terms used herein shall bear the meaning as understood by a person who is ordinarily skilled in the art. Any ranges given herein include both the lower and the upper endpoints of the range. Furthermore, the present invention covers all possible combinations of particular and preferred embodiments described herein.

In one embodiment, the present invention provides a stable topical pharmaceutical composition comprising fertility aid or pharmaceutically acceptable salts thereof, wherein the said composition is a cream, an ointment, a lotion or a gel.

In one the preferred embodiment, the present invention provides a stable topical pharmaceutical composition comprising fertility aid or pharmaceutically acceptable salts thereof, wherein the said composition is topical solution of solution for spray dosage from.

The term “stable pharmaceutical composition” of the present invention means the topical composition retains consistency as a gel, solution of solution for spray and the individual assay of fertility aid in the said composition is at least 95% when stored at 25° C./60% RH for at least one month, preferably for at least 2 months, and more preferably for at least 3 months. The abbreviation “RH” stands for relative humidity corresponding to the storage conditions. The individual assay of the active ingredient can be carried out by any conventional analytical methods, preferably by HPLC method. The HPLC (High-performance liquid chromatography) technique in analytical chemistry is used to separate, identify, and quantify each component in a mixture. The HPLC method is well known in the art for the quantification and qualification of analytes.

The term “about” means within an acceptable error range for the particular value of ±10% or as determined by one of ordinary skill in the art, which will depend in part on how the value is measured or determined, i.e., the limitations of the measurement system.

The term “infertility” in the present invention means a couple has been having unprotected intercourse for a year or more-based on the female's age and pregnancy does not occur. The infertility is identified and unidentified type depending on the identity of the cause of infertility.

The term “identified infertility” in the present invention means the cause of infertility in men or female can be identified by medical examination or clinical test.

The term “unidentified infertility” in the present invention means the results of medical examination or clinical test for measurement of infertility is normal still the male not able to make female pregnant or female not able to get pregnant through unprotected sex. Causes of unidentified infertility mainly includes insufficient cervical mucus, vaginal dryness, poor egg quality, egg is not released at the optimum time for fertilization, sperm may not be able to reach the egg, fertilization may fail to occur, transport of the zygote may be disturbed, or implantation fails.

The term “trying to conceive” in the present invention means women or men having regular, unprotected intercourse whether it is intentional or not.

The term “sperm penetration assay” or “sperm migration test” in the present invention means the test to predict the capacity of a man's sperm to fertilize a woman's egg.

The term “spermatozoa motility” or “sperm motility” of the present invention means the ability of sperm to move efficiently.

The term “Spermatozoa vitality test” or “sperm vitality test” of the present invention means the percentage of live sperm in the semen sample.

The term “Spermatozoa morphology test” or “sperm morphology test” of the present invention means analysis of the size and shape of sperm as part of a semen analysis to evaluate male infertility. Sperm morphology results are reported as the percentage of sperm that appear normal when semen is viewed under a microscope.

The term “hypo-osmotic swelling test” or “HOS test” of the present invention means assay used to evaluate the functional integrity of the sperm's plasma membrane. It serves as a useful indicator of fertility potential of sperm.

The term “adjuvant therapy” or “adjunct therapy” of the present invention means therapy that is given separately or simultaneously in addition to the primary or initial therapy to maximize its effectiveness.

The term “fertility aid” of the present invention means a single agent or supplement or group of agents or supplements that collectively boost fertility in males, females, or both by providing or enhancing the atmosphere for the sperm to enter in to the ovum.

In a preferred embodiment, the fertility aid is selected from the group consisting of sodium hyluronate, L arginine, Vitamins, carbohydrate, electrolytes, extract or mixture thereof. The sodium hyluronate is high molecular weight, medium molecular weight, low molecular weight, oligomer (very low molecular weight) or mixture thereof. High molecular weight sodium hyluronate has 1600 kilo Dalton to 2000 kilo Dalton molecular weight. Medium molecular weight sodium hyluronate has 500 kilo Dalton to 1600 kilo Dalton molecular weight. Low molecular weight sodium hyluronate has 8 kilo Dalton to 200 kilo Dalton molecular weight. Oligomer or very low molecular weight sodium hyluronate has <=8 kilo Dalton molecular weight

In one of the embodiment, the sodium hyluronate has molecular weight greater than 800 kilo Dalton, greater than 700 kilo Dalton, greater than 600 kilo Dalton, greater than 500 Kilo Dalton, greater than 400 kilo Dalton, greater than 300 kilo Dalton, greater than 200 kilo Dalton, greater than 100 kilo Dalton, greater than 50 kilo Dalton, greater than 5 kilo Dalton, greater than 1 kilo Dalton or mixture thereof.

In one of the embodiment of present invention provides a pharmaceutical composition having mixture of oligomer or very low molecular weight sodium hyluronate and low molecular weight sodium hyluronate.

In one of the embodiment of present invention provides a stable topical pharmaceutical composition having mixture of high molecular weight, medium molecular weight, low molecular weight and oligomer sodium hyluronate.

In one of the embodiment of present invention provides a stable topical pharmaceutical composition comprising sodium hyluronate having high molecular weight, medium molecular weight, low molecular weight, oligomer or mixture thereof.

The vitamins can be selected from the group comprising of niacinamide, acetyl L-carnitine, Vitamin

B, Vitamin C, Vitamin D, Vitamin E or mixture thereof. The said vitamin presents in a concentration of at least about % w/w to about 10% w/w of the said pharmaceutical composition.

The electrolytes can be selected from the group comprising of Sodium chloride, Potassium phosphate, Sodium phosphate, Magnesium chloride, Calcium chloride, Sodium bicarbonate or mixture thereof. The said vitamin presents in a concentration of at least about 0.01% w/w to about 30% w/w of the said pharmaceutical composition.

The carbohydrate can be selected from the group comprising of Galactose, fructose, Glucose or mixture thereof. The said carbohydrate present in a concentration of at least about 0.1% w/w to about 15% w/w of the said pharmaceutical composition.

The extract can be plant derived or animal derived. The plant derived extracted can be selected from the group comprising of tribulus terrestris extract, Aloe vera, panax ginseng extract or mixture thereof. The animal derived extracted can be selected from the group comprising of Bovine mucin. The said extract present in a concentration of at least about 0.01% w/w to about 15% w/w of the said pharmaceutical composition.

In a preferred embodiment, the concentration of fertility aid or its pharmaceutically acceptable salt is at least about 0.001 to about 50% w/w of the total composition. In one embodiment, the concentration of fertility aid is at least about 1 to about 40% w/w with respect to the total weight of the composition. In a preferred embodiment, the concentration of fertility aid is at least about 2.5 to about 30% w/w. The active ingredients used in the stable composition comprises of fertility aid or pharmaceutically acceptable salts thereof. Further, the amount of the fertility aid in the formulation can be varied that is within the scope of a person skilled in the art.

The term “pharmaceutically acceptable excipient” (also named as “excipients”) refers to a substance formulated alongside with the active pharmaceutical ingredient of a medicinal product and includes all kind of pharmaceutically acceptable compounds commonly used in pharmaceutical compositions and in particular gel, solution or solution for spray composition. The term “pharmaceutically acceptable excipients” comprise gelling agent, soothing agent, preservative, antioxidant, weeting agent, solubilizers, humectant, buffering agent, surfactants, moisturizing agent, emollients, chelating agents, solvents and mixtures thereof

The stable composition of the present invention can comprise pharmaceutically acceptable excipients selected from a gelling agent, soothing agent, preservative, antioxidant, weeting agent, solubilizers, humectant, buffering agent, surfactants, moisturizing agent, emollients, chelating agents, solvents or mixture thereof.

The gelling agents can be selected from the group comprising of aluminum monostearate, gelatin, glyceryl monooleate, glyceryl palmitostearate, pectin, zinc acetate, carbomer 934, carboxy methyl cellulose, hydroxy propyl cellulose, arabinogalactan, Guar gum, carrageenan, xanthan gum or mixture thereof. The said gelling agent present in a concentration of at least about 1% w/w to about 20% w/w of the said pharmaceutical composition.

The soothing agents can be selected from the group comprising of aloe vera, allantoin and rose hip oil aloe vera, allantoin and rose hip oil. The said gelling agent present in a concentration of at least about 1% w/w to about 30% w/w of the said pharmaceutical composition.

The preservatives can be selected from the group comprising of Alcohol, Benzalkonium Chloride, Benzethonium Chloride, Benzoic Acid, Benzyl Alcohol, Boric Acid, Bronopol, Butylene Glycol, Butylparaben, Calcium Acetate, Calcium Chloride, Calcium Lactate, Cetrimide, Cetylpyridinium Chloride, Chlorhexidine, Chlorobutanol, Chlorocresol, Chloroxylenol, Citric Acid Monohydrate, Cresol, Ethylparaben, Glycerin, Hexetidine, Imidurea, Galguard Trident, Methylparaben, Pentetic Acid, Phenol, Phenoxyethanol, Phenylethyl Alcohol, Phenylmercuric Acetate, Phenylmercuric Borate, Potasium sorbate, Germal plus, Phenylmercuric Nitrate, Potassium Benzoate, Potassium Metabisulfite, Potassium Sorbate, Propionic Acid, Propylene Glycol, Propylparaben, Propylparaben Sodium, Sodium Acetate, Sodium Benzoate, Sodium Borate, Sodium Lactate, Sodium Metabisulfite, Sodium Propionate, Sodium Sulfite, Sorbic Acid, Sulfur Dioxide, Thimerosal, Nipaguard CG, Nipaguard PO5, Germall plus or mixture thereof. The said preservatives present in a concentration of at least about 0.01% w/w to about 10% w/w of the said pharmaceutical composition.

The antioxidants can be selected from the group comprising of Alpha Tocopherol, Ascorbic Acid, Ascorbyl Palmitate, Butylated Hydroxyanisole, Butylated Hydroxytoluene, Citric Acid Monohydrate, Erythorbic Acid, Fumaric Acid, Malic Acid, Methionine, Potassium Metabisulfite, Propionic Acid, Propyl Gallate, Sodium Ascorbate, Sodium Formaldehyde Sulfoxylate, Sodium Metabisulfite, Sodium Sulfite, Sodium Thiosulfate, Sulfur Dioxide, Thymol, Vitamin E Polyethylene Glycol Succinate or mixture thereof. The said antioxidants present in a concentration of at least about 0.01% w/w to about 10% w/w of the said pharmaceutical composition.

The wetting agents can be selected from the group comprising of Benzalkonium Chloride, Benzethonium chloride, Cetylpyridinium Chloride, Docusate Sodium, Glycine, Glycofurol, Hypromellose, Poloxamer, Phospholipids, Polyoxyethylene Alkyl Ethers, Polyoxyethylene Castor Oil Derivatives, Polyoxyethylene Sorbitan Fatty Acid Esters, Polyoxyethylene Stearates, Sodium Lauryl Sulfate, Sorbitan Esters (Sorbitan Fatty Acid Esters), propylene glycol, Caprylyl glycol, Tricaprylin or mixture thereof. The said wetting agents present in a concentration of at least about 0.01% w/w to about 10% w/w of the said pharmaceutical composition.

The solubilizers can be selected from the group comprising of Benzalkonium Chloride, Benzyl Benzoate, Cetylpyridinium Chloride, Cyclodextrins, Glyceryl Monostearate, Hydroxypropyl Betadex, Hypromellose, Lecithin, Macrogol 15 Hydroxystearate, Phospholipids, Poloxamer, Polyoxyethylene Alkyl Ethers, Polyoxyethylene Castor Oil Derivatives, Polyoxyethylene Sorbitan Fatty Acid Esters, Polyoxyethylene Stearates, Polyoxylglycerides, Pyrrolidone, Sorbitan Esters (Sorbitan Fatty Acid Esters), Stearic Acid, Sulfobutylether b-Cyclodextrin, Tricaprylin, Triolein, Vitamin E Polyethylene Glycol Succinate, Wax Anionic Emulsifying, Wax Nonionic Emulsifying or mixture thereof. The said solubilizers present in a concentration of at least about 0.01% w/w to about 10% w/w of the said pharmaceutical composition.

The humectant can be selected from the group comprising of Ammonium Alginate, Butylene Glycol, Cyclomethicone, Glycerin, Polydextrose, Propylene Glycol, Caprylyl glycol, 1,2 propendiol, Sodium Hyaluronate, Sodium Lactate, Sorbitol, Triacetin, Trehalose, Xylitol or mixture thereof. The said humectant present in a concentration of at least about 0.01% w/w to about 15% w/w of the said pharmaceutical composition.

The buffering agents can be selected from the group comprising of Adipic Acid, Boric Acid, Calcium Carbonate, Calcium Lactate, Tribasic Calcium Phosphate, Citric Acid Monohydrate, Glycine, Maleic Acid, Malic Acid, Methionine, Monosodium, Glutamate, Potassium Citrate, Sodium Acetate, Sodium Borate, Sodium Carbonate, Sodium Citrate Dihydrate, Sodium Hydroxide, Sodium Lactate, Dibasic Sodium Phosphate, Sodium Phosphate, Potasium phosphate or mixture thereof.

The surfactants can be selected from the group comprising of Cetrimide, Cetylpyridinium Chloride, Docusate Sodium, Glyceryl Monooleate, Lauric Acid, Macrogol 15 Hydroxystearate, Myristyl Alcohol, Phospholipids, Polyoxyethylene Sorbitan Fatty Acid Esters, Polyoxylglycerides, Sodium Lauryl Sulfate, Sorbitan Esters (Sorbitan Fatty Acid Esters), Vitamin E Polyethylene Glycol Succinate or mixture thereof. The said surfactants present in a concentration of at least about 0.01% w/w to about 15% w/w of the said pharmaceutical composition.

The emollients can be selected from the group comprising of Almond oil, Aluminum monostearate, Canola oil, Castor oil, Cetostearyl alcohol, Cholesterol, Coconut oil, Cyclomethicone, Dimethicone, Ethylene glycol stearates, Glycerin, Glyceryl monooleate, Glyceryl monostearate, Isopropyl myristate, Isopropyl palmitate, Lecithin, Mineral oil, Mineral oil, Light, Mineral oil And Lanolin alcohols, Myristyl alcohol, Octyldodecanol, Oleyl alcohol, Petrolatum, Petrolatum and Lanolin alcohols, Safflower oil, Sunflower oil, Tricaprylin, Triolein, Wax cetyl esters, Xylitol, Zinc acetate or mixture thereof. The said emollients present in a concentration of at least about 0.01% w/w to about 5% w/w of the said pharmaceutical composition.

The chelating agents can be selected from the group comprising of calcium acetate, hydroxypropyl betadex, potassium citrate, citric acid, citric acid monohydrate, disodium edetate, edetic acid, malic acid, pentetic acid, phosphoric acid, sodium citrate dihydrate, dibasic sodium phosphate, monobasic sodium phosphate, tartaric acid, potassium citrate, fumaric acid, maltol, pentetic acid or mixture thereof. The said chelating agent present in a concentration of at least about 0.01% w/w to about 5% w/w of the said pharmaceutical composition.

The Solvent can be selected from the group comprising of Purified Water, Arometic water like Campher water, Alcohol, Glycerol, Propylene Glycol, Ether, Fixed oil form plant source or mixture thereof.

In another embodiment of the present invention provides a stable topical pharmaceutical composition comprising Sodium hyluronate, Panax ginseng extract, pharmaceutically acceptable excipients or mixture thereof.

In another embodiment of the present invention provides a stable topical pharmaceutical composition comprising Sodium hyluronate, Panax ginseng extract, pharmaceutically acceptable excipients or mixture thereof, wherein said composition have glucuronic acid, wherein said glucuronic acid is present in at least about 46% w/w.

In another embodiment of the present invention provides a stable topical pharmaceutical composition comprising Sodium hyluronate, Panax ginseng extract, pharmaceutically acceptable excipients or mixture thereof, wherein said composition have Hyaluronic Acid, wherein said Hyaluronic Acid is present in at least about 95% w/w.

In another embodiment of the present invention provides a stable topical pharmaceutical composition comprising Sodium hyluronate, Panax ginseng extract, pharmaceutically acceptable excipients or mixture thereof, wherein said composition have heavy metal content not more than 20 parts per million.

In another embodiment of the present invention provides a stable topical pharmaceutical composition comprising Sodium hyluronate, Panax ginseng extract, pharmaceutically acceptable excipients or mixture thereof, wherein said composition have lead content not more than 3 parts per million.

In another embodiment of the present invention provides a stable topical pharmaceutical composition comprising Sodium hyluronate, Panax ginseng extract, pharmaceutically acceptable excipients or mixture thereof, wherein said composition have arsenic content not more than 2 parts per million.

In another embodiment of the present invention provides a stable topical pharmaceutical composition comprising Sodium hyluronate, Panax ginseng extract, pharmaceutically acceptable excipients or mixture thereof, wherein said composition have cadmium content not more than 1 parts per million.

In another embodiment of the present invention provides a stable topical pharmaceutical composition comprising Sodium hyluronate, Panax ginseng extract, pharmaceutically acceptable excipients or mixture thereof, wherein said composition have mercury content not more than 0.1 parts per million.

In another embodiment of the present invention provides a stable topical pharmaceutical composition comprising fertility aid or pharmaceutically acceptable salt thereof and pharmaceutically acceptable excipients, wherein said composition have heavy metal content not more than 20 parts per million.

In another embodiment of the present invention provides a stable topical pharmaceutical composition comprising fertility aid or pharmaceutically acceptable salt thereof and pharmaceutically acceptable excipients, wherein said composition have lead content not more than 3 parts per million.

In another embodiment of the present invention provides a stable topical pharmaceutical composition comprising fertility aid or pharmaceutically acceptable salt thereof and pharmaceutically acceptable excipients, wherein said composition have arsenic content not more than 2 parts per million.

In another embodiment of the present invention provides a stable topical pharmaceutical composition comprising fertility aid or pharmaceutically acceptable salt thereof and pharmaceutically acceptable excipients, wherein said composition have cadmium content not more than 1 parts per million.

In another embodiment of the present invention provides a stable topical pharmaceutical composition comprising fertility aid or pharmaceutically acceptable salt thereof and pharmaceutically acceptable excipients, wherein said composition have mercury content not more than 0.1 parts per million.

In another embodiment of the present invention provides a stable topical pharmaceutical composition comprising Sodium hyluronate, Aloe vera powder, Panax ginseng extract, Fructose, 1,3-Propendiol, Germall plus, pharmaceutically acceptable excipients or mixture thereof.

In another embodiment of the present invention provides a stable topical pharmaceutical composition comprising Sodium hyluronate oligo grade molecular weight less than 8 kilo Dalton, Sodium hyluronate low molecular weight less than 8 kilo Dalton to 200 kilo Dalton, Sodium hyluronate Medium molecular weight less than 500 kilo Dalton to 1600 kilo Dalton, Sodium hyluronate high molecular weight less than 1500 kilo Dalton to 2000 kilo Dalton, Aloe vera powder, Panax ginseng extract, Fructose, 1,3 Propendiol, Germall plus, pharmaceutically acceptable excipients or mixture thereof.

In another embodiment of the present invention provides a stable topical pharmaceutical composition comprising Sodium hyluronate oligo grade molecular weight less than 8 kilo Dalton, Sodium hyluronate low molecular weight less than 8 kilo Dalton-200 kilo Dalton, Aloe vera powder, Panax ginseng extract, Fructose, 1,3 Propendiol, Germall plus, pharmaceutically acceptable excipients or mixture thereof.

In another embodiment of the present invention provides a stable topical pharmaceutical composition comprising Bovine mucin, Niacinamide, arabinogalactan, Galactose, Galguard Trident, 1,3 Propendiol, Glucose, pharmaceutically acceptable excipients or mixture thereof.

In another embodiment of the present invention provides a stable topical pharmaceutical composition comprising Tribulus terrestris extract, L arginine, Allantoin, Guar gum/caragenaan, Potasium sorbate, Phenoxyethanol, Galactose, Fructose, 1,3 Propendiol, pharmaceutically acceptable excipients or mixture thereof.

In another embodiment of the present invention provides a stable topical pharmaceutical composition comprising fertility aid or pharmaceutically acceptable salt thereof and pharmaceutically acceptable excipients, wherein said topical composition is gel composition.

In another embodiment of the present invention provides a stable topical pharmaceutical composition comprising fertility aid or pharmaceutically acceptable salt thereof and pharmaceutically acceptable excipients, wherein said topical composition is solution or solution for spray composition.

In another embodiment of the present invention provides a stable topical pharmaceutical composition comprising Sodium hyluronate, Panax ginseng extract, pharmaceutically acceptable excipients or mixture thereof, wherein said composition is gel composition.

In another embodiment of the present invention provides a stable topical pharmaceutical composition comprising Sodium hyluronate, Panax ginseng extract, pharmaceutically acceptable excipients or mixture thereof, wherein said composition is solution or solution for spray composition.

In another embodiment of the present invention provides a process for the preparation of a stable topical composition, wherein the said process comprises the following steps:
a. Transfer required quantity of solvent in a vessel;
b. Optionally, heat the solvent between 60-65° C. with continuous stirring;
c. Add required quantity of gelling agent, soothing agent, preservative, antioxidant, weeting agent, solubilizers, humectant, buffering agent, surfactants, moisturizing agent and solvents;
d. Stir the mixture till transparent gel obtain;
e. Optionally, sonicate the gel obtain in step d.

Wherein said process provides pharmaceutical composition in gel dosage form.

In another embodiment of the present invention provides a process for the preparation of a stable topical composition, wherein the said process comprises the following steps:
a. Transfer required quantity of solvent in a vessel;
b. Optionally, heat the solvent between 60-65° C. with continuous stirring;
c. Add required quantity of gelling agent, soothing agent, preservative, antioxidant, weeting agent, solubilizers, humectant, buffering agent, surfactants, moisturizing agent and solvents;
d. Stir the mixture till transparent solution is obtain.

Wherein said process provides pharmaceutical composition in solution dosage form.

In another embodiment of the present invention provides a process for the preparation of a stable topical composition, wherein the said process comprises the following steps:
a. Transfer required quantity of solvent in a vessel;
b. Optionally, heat the solvent between 60-65° C. with continuous stirring;
c. Add required quantity of gelling agent, soothing agent, preservative, antioxidant, weeting agent, solubilizers, humectant, buffering agent, surfactants, moisturizing agent and solvents;
d. Stir the mixture till transparent solution is obtain,
e. Fill the solution in to container to obtain spray.

Wherein said process provides pharmaceutical composition in solution for spray dosage form.

In preferred embodiment of the present invention provides a process for the preparation of a stable topical composition, wherein the said process comprises following the steps:
a. Transfer required quantity of water in a vessel;
b. Optionally, heat the solvent between 60-65° C. with continuous stirring;
c. Add required quantity of gelling agent, soothing agent, preservative, antioxidant, weeting agent, solubilizers, humectant, buffering agent, surfactants, moisturizing agent and solvents;
d. Stir the mixture till transparent gel obtain;

In another embodiment of the present invention provides a stable topical pharmaceutical composition comprising fertility aid or pharmaceutically acceptable salts thereof and pharmaceutically acceptable excipients, wherein the said composition is in gel or solution or solution for spray form, where in said composition is filled into single layer or multilayer laminated aluminum collapsible tube, spray pump, single use cartridge seamless plastic tube, elongated nozzle tube tube with suitable applicator packaging container.

In another embodiment of the present invention provides a stable topical pharmaceutical composition comprising fertility aid or pharmaceutically acceptable salts thereof and at least one pharmaceutically acceptable excipient, wherein the pH of the said composition is about more than 6. In a preferred embodiment, the pH of the composition is about 7. In a preferred embodiment, the pH of the composition is about 7 to about 9. In another preferred embodiment, the pH of the composition is about 7.2 to about 8.8. In a more preferred embodiment, the pH of the composition is about 8.5.

In another embodiment of the present invention provides a stable topical pharmaceutical composition comprising fertility aid or pharmaceutically acceptable salts thereof and pharmaceutically acceptable excipients, wherein the Osmolality of the present composition is about 200 mOsmo/kg to 600 mOsmo/kg.

In another embodiment of the present invention provides a stable topical pharmaceutical composition comprising fertility aid or pharmaceutically acceptable salts and pharmaceutically acceptable excipients, thereof wherein the apparent viscosity of the present invention is about 2000 centipoise to 14,000 centipoise.

In another embodiment of the present invention provides a stable topical pharmaceutical composition comprising fertility aid or pharmaceutically acceptable salts thereof and pharmaceutically acceptable excipients, wherein said composition does not contain E. coli, Salmonella, Staphylococcus aureus, or Pseudomonas aeruginosa.

In another embodiment of the present invention provides a stable topical pharmaceutical composition comprising fertility aid or pharmaceutically acceptable salts and pharmaceutically acceptable excipients, thereof wherein said composition does not contain more than 10 colony forming unit per gram of yeast and molds.

In another embodiment of the present invention provides a stable topical pharmaceutical composition comprising fertility aid or pharmaceutically acceptable salts thereof and pharmaceutically acceptable excipients, wherein the said composition is filled in squeezable low density polyethylene container.

In another embodiment of the present invention provides a stable topical pharmaceutical composition comprising fertility aid or pharmaceutically acceptable salts thereof and pharmaceutically acceptable excipients, wherein individual assay of fertility aid in the said composition is at least 95% when stored at 25° C./60% RH for at least one month, preferably for at least 2 months, and more preferably for at least 3 months.

In another embodiment of the present invention provides a stable topical pharmaceutical composition comprising fertility aid or pharmaceutically acceptable salts thereof and pharmaceutically acceptable excipients, wherein individual assay of fertility aid in the said composition is at least 95% when stored at 40° C./75% RH for at least one month, preferably for at least 2 months, and more preferably for at least 3 months.

In another embodiment of the present invention provides a stable topical pharmaceutical composition comprising fertility aid or pharmaceutically acceptable salts thereof and pharmaceutically acceptable excipients, wherein the said composition is in gel or solution or solution for spray form, wherein the said composition is sufficiently resembling human cervical mucus of vagina.

In another embodiment of the present invention provides a stable topical pharmaceutical composition comprising fertility aid or pharmaceutically acceptable salts thereof and pharmaceutically acceptable excipients, wherein the said composition is in gel or solution or solution for spray form, wherein the said composition has pH 6 to 7.5 which resembling human cervical mucus pH of vagina.

In another embodiment of the present invention provides a stable topical pharmaceutical composition comprising fertility aid or pharmaceutically acceptable salts thereof and pharmaceutically acceptable excipients, wherein the said composition is in gel or solution or solution for spray form, wherein the said composition has pH 6 to 7.5 which resembling human semen fluid pH.

In another embodiment of the present invention provides a stable topical pharmaceutical composition comprising fertility aid or pharmaceutically acceptable salts thereof and pharmaceutically acceptable excipients, wherein the said composition is in gel or solution or solution for spray form, wherein the said composition has Osmolarity about 269 mOsmo/kg to about 340 mOsmo/kg which resembling Osmolarity of human semen fluid and cervical mucus.

In another embodiment, the present invention relates to a stable topical pharmaceutical composition comprising fertility aid or pharmaceutically acceptable salts thereof and pharmaceutically acceptable excipients, wherein the said composition is in gel or solution or solution for spray form, wherein the said fertility aid or pharmaceutically acceptable salts thereof does not penetrate into systemic circulation, wherein the said fertility aid or pharmaceutically acceptable salts thereof penetrate into systemic circulation in non-detectable amount.

In another embodiment, the present invention relates to a stable topical pharmaceutical composition comprising fertility aid or pharmaceutically acceptable salts thereof and pharmaceutically acceptable excipients, wherein the said composition is in gel or solution or solution for spray form, wherein the said topical composition provides irritation free effect.

In another embodiment of the present invention, a stable topical pharmaceutical composition comprising fertility aid or pharmaceutically acceptable salts thereof and pharmaceutically acceptable excipients, wherein the said composition is in gel or solution or solution for spray form, wherein the said composition can be used for female or male administration for adjuvant therapy or supportive aid of infertility, wherein the said infertility is identified or unidentified in men, women or both.

In another embodiment of the present invention, a stable topical pharmaceutical composition comprising fertility aid or pharmaceutically acceptable salts thereof and pharmaceutically acceptable excipients, wherein the said composition is in gel or solution or solution for spray form, wherein the said composition can be used by trying to conceive female or male for adjuvant therapy or supportive aid of infertility, wherein the said infertility is identified or unidentified in men, women or both.

In another embodiment of the present invention, a stable topical pharmaceutical composition comprising fertility aid or pharmaceutically acceptable salts thereof and pharmaceutically acceptable excipients, wherein the said composition is in gel or solution or solution for spray form, wherein the said composition can be used by trying to conceive female or male for adjuvant therapy or supportive aid of infertility, wherein the said composition is use separately or simultaneously in the treatment of infertility in men, women or both.

In another embodiment of the present invention provides a stable topical pharmaceutical composition comprising fertility aid or pharmaceutically acceptable salts thereof and pharmaceutically acceptable excipients, wherein the said composition is in gel or solution or solution for spray form, wherein the said composition is applied in vagina or on penis by the trying to conceive patients or patients having infertility in men, women or both.

In another embodiment of the present invention provides a stable topical pharmaceutical composition comprising fertility aid or pharmaceutically acceptable salts thereof and pharmaceutically acceptable excipients, wherein the said composition is in gel or solution or solution for spray form, wherein the said composition provides optimum physiological environment for semen or sperm in vagina.

In another embodiment of the present invention provides a stable topical pharmaceutical composition comprising fertility aid or pharmaceutically acceptable salts thereof and pharmaceutically acceptable excipients, wherein the said composition is in gel or solution or solution for spray form, wherein the said composition provides physiological property and environment to semen or sperm which is similar to cervical mucus of vagina.

In another embodiment, the present invention relates to a stable topical pharmaceutical composition comprising fertility aid or pharmaceutically acceptable salts thereof and pharmaceutically acceptable excipients, wherein the said composition can be used to avoid vaginal dryness and provide lubricating effect thereby prevent painful intercourse.

In another embodiment of the present invention provides a stable topical pharmaceutical composition comprising fertility aid or pharmaceutically acceptable salts thereof and pharmaceutically acceptable excipients, wherein the said composition is used in identified infertility and/or unidentified infertility in men, women or both.

In another embodiment of the present invention provides a stable topical pharmaceutical composition comprising fertility aid or pharmaceutically acceptable salts thereof and pharmaceutically acceptable excipients, wherein the said composition provides equivalent or greater percentage of sperm penetration assay, Spermatozoa/sperm motility, Spermatozoa/sperm vitality, Spermatozoa/Sperm Morphology and Hypo-osmotic swelling.

In another embodiment of the present invention provides a stable topical pharmaceutical composition comprising fertility aid or pharmaceutically acceptable salts and pharmaceutically acceptable excipients, thereof wherein the said composition provides at least at least 85% of Spermatozoa/sperm motility in 20 minute compare to initial Spermatozoa/sperm motility value, at least 55% of Spermatozoa/sperm motility in 120 minute compare to initial Spermatozoa/sperm motility value and at least 35% of Spermatozoa/sperm motility in 240 minute compare to initial Spermatozoa/sperm motility value.

In another embodiment of the present invention provides a stable topical pharmaceutical composition comprising fertility aid or pharmaceutically acceptable salts thereof and pharmaceutically acceptable excipients, wherein the said composition provides at least 85% of Spermatozoa/sperm vitality in 20 minute compare to initial Spermatozoa/sperm vitality value, at least 55% of Spermatozoa/sperm vitality in 120 minute compare to initial Spermatozoa/sperm vitality value and at least 35% of Spermatozoa/sperm vitality in 240 minute compare to initial Spermatozoa/sperm vitality value.

In another embodiment of the present invention provides a stable topical pharmaceutical composition comprising fertility aid or pharmaceutically acceptable salts thereof and pharmaceutically acceptable excipients, wherein the said composition provides at least 85% of Spermatozoa/sperm Morphology in 20 minute compare to initial Spermatozoa/sperm Morphology value, at least 55% of Spermatozoa/sperm Morphology in 120 minute compare to initial Spermatozoa/sperm Morphology value and at least 35% of Spermatozoa/sperm Morphology in 240 minute compare to initial Spermatozoa/sperm Morphology value.

In another embodiment of the present invention provides a stable topical pharmaceutical composition comprising fertility aid or pharmaceutically acceptable salts thereof and pharmaceutically acceptable excipients, wherein the said composition provides at least 85% of Hypo-osmotic swelling in 20 minute compare to initial Hypo-osmotic swelling value, at least 55% of Hypo-osmotic swelling in 120 minute compare to initial Hypo-osmotic swelling value and at least 35% of Hypo-osmotic swelling in 240 minute compare to initial Hypo-osmotic swelling value.

### EXAMPLES

The present invention has been described by way of example only, and it is to be recognized that modifications thereto falling within the scope and spirit of the appended claims, and which would be obvious to a person skilled in the art based upon the disclosure herein, are also considered to be included within the scope of this invention.

Example-1: General composition

| Sr. | | Ingredients |
|---|---|---|
| No. | Ingredient Category | % w/w |
| 1 | Fertility Aid | 0.01-50% |
| 2 | Gelling agent | 1-20% |
| 3 | Soothing agents | 1-30% |
| 4 | Preservatives | 0.01-10% |
| 5 | Antioxidants | 0.01-10% |
| 6 | Wetting agents | 0.01-10% |
| 7 | Solubilizers | 0.01-10% |
| 8 | Humectant | 0.01-15% |
| 9 | Buffering agent | q.s. |
| 10 | Surfactants | 0.01-15% w/w |
| 11 | Solvent | q.s. |

### Manufacturing Process

a) Optionally, heat the solvent between 60-65° C. with continuous stirring;
b) Add required quantity of gelling agent, soothing agent, preservative, antioxidant, weeting agent, solubilizers, humectant, buffering agent, surfactants, moisturizing agent and solvents;
c) Stir the mixture till transparent gel obtain;
d) Optionally, sonicate the gel obtain in step d.

Example 2:

| Sr. | | Ingredients |
|---|---|---|
| No. | Ingredient Name | (% w/w) |
| 1 | Sodium hyluronate | 1 |
| 2 | Aloe vera powder | 0.5 |
| 3 | Panax ginseng extract | 1 |
| 4 | Sodium chloride | 0.25 |
| 5 | Potassium phosphate | 0.25 |
| 6 | Sodium phosphate | 0.25 |
| 7 | Magnesium chloride | 0.2 |
| 8 | Calcium chloride | 0.2 |
| 9 | Sodium bicarbonate | 0.2 |
| 10 | Fructose | 0.5 |
| 11 | 1,3 Propendiol | 3 |
| 12 | Germall plus | 0.5 |
| 13 | Purified water | 92.15 |

### Manufacturing Process

a) Optionally, heat the water between 60-65° C. with continuous stirring;
b) Add Sodium hyluronate, Aloe vera powder, Panax ginseng extract, Sodium chloride, Potassium phosphate, Sodium phosphate, Magnesium chloride, Calcium chloride, Sodium bicarbonate, Fructose, 1,3 Propendiol, Germall plus;
c) Stir the mixture till transparent gel obtain;
d) Optionally, sonicate the gel obtain in step d.

Example 3:

| Sr. | | Ingredients |
|---|---|---|
| No. | Ingredient Name | (% w/w) |
| 1 | Tribulus terrestris | 0.5 |
| | extract | |
| 2 | L arginine | 0.5 |
| 3 | Allantoin | 0.2 |
| 4 | Guar gum/caragenaan | 0.5 |
| 5 | Potasium sorbate | 0.2 |
| 6 | Phenoxyethanol | 0.2 |
| 7 | Sodium chloride | 0.25 |
| 8 | Potasium phosphate | 0.25 |
| 9 | Sodium phosphate | 0.25 |
| 10 | Galactose | 0.25 |
| 11 | Fructose | 0.25 |
| 12 | 1,3 Propendiol | 3 |
| 13 | Purified water | 93.65 |

### Manufacturing Process

a) Optionally, heat the water between 60-65° C. with continuous stirring;
b) Add tribulus terrestris extract, L arginine, allantoin, guar gum/caragenaan, potasium sorbate, phenoxyethanol, sodium chloride, potasium phosphate, sodium phosphate, galactose, fructose, 1,3 propendiol;
c) Stir the mixture till transparent gel obtain;
d) Optionally, sonicate the gel obtain in step d.

Example 4:

| Sr. | | Ingredient |
|---|---|---|
| No. | Ingredient Name | % w/w |
| 1 | Bovine mucin | 0.5 |
| 2 | Niacinamide | 1 |
| 3 | Arabinogalactan | 0.5 |
| 4 | Sodium chloride | 0.25 |
| 5 | Potasium phosphate | 0.25 |
| 6 | Sodium phosphate | 0.25 |
| 7 | Galactose | 0.25 |
| 8 | Calcium chloride | 0.2 |
| 9 | Sodium bicarbonate | 0.2 |
| 10 | Galguard Trident | 0.5 |
| 11 | 1,3 Propendiol | 3 |
| 12 | Glucose | 0.25 |
| 13 | Purified water | 92.85 |

### Manufacturing Process

a) Optionally, heat the water between 60-65° C. with continuous stirring;
b) Add bovine mucin, niacinamide, arabinogalactan, sodium chloride, potassium phosphate, sodium phosphate, galactose, calcium chloride, sodium bicarbonate, galguard trident, 1,3 propendiol, glucose, purified water;
c) Stir the mixture till transparent gel obtain;
d) Optionally, sonicate the gel obtain in step d.

Example 5:

| Sr. | | Ingredients |
|---|---|---|
| No. | Ingredient | (% w/w) |
| 1 | Sodium hyluronate oligo grade Molecular | 0.5 |
| | weight less than 8 kilo Dalton | |
| 2 | Sodium hyluronate low Molecular weight | 0.5 |
| | less than 8 kilo Dalton-200 kilo Dalton | |
| 4 | Aloe vera powder | 0.5 |
| 5 | Panax ginseng extract | 1 |
| 6 | Sodium chloride | 0.25 |
| 7 | Potasium phosphate | 0.25 |
| 8 | Sodium phosphate | 0.25 |
| 9 | Magnesium chloride | 0.2 |
| 10 | Calcium chloride | 0.2 |
| 11 | Sodium bicarbonate | 0.2 |
| 12 | Fructose | 0.5 |
| 13 | 1,3 Propendiol | 3 |
| 14 | Germall plus | 0.5 |
| 16 | Purified water | q.s. |

### Manufacturing Process

a) Optionally, heat the water between 60-65° C. with continuous stirring;
b) Add Sodium hyluronate oligo grade Molecular weight less than 8 kilo Dalton, Sodium hyluronate low Molecular weight less than 8 kilo Dalton-200 kilo Dalton, Aloe vera powder, Panax ginseng extract, Sodium chloride, Potasium phosphate, Sodium phosphate, Magnesium chloride, Calcium chloride, Sodium bicarbonate, Fructose, 1,3 Propendiol, Germall plus, Purified water;
c) Stir the mixture till transparent solution is obtained;
d) Optionally, fill the solution in to container to obtain spray.

Example 6:

| Sr. | | Ingredients |
|---|---|---|
| No. | Ingredient | (% w/w) |
| 1 | Sodium hyluronate oligo grade | 0.3 |
| | molecular weight less than 8 kilo Dalton | |
| 2 | Sodium hyluronate low molecular weight | 0.3 |
| | less than 8 kilo Dalton - 200 kilo Dalton | |
| 3 | Sodium hyluronate Medium molecular weight | 0.3 |
| | less than 500 kilo Dalton to 1600 kilo Dalton | |
| 4 | Sodium hyluronate high molecular weight less | 0.3 |
| | than 1500 kilo Dalton to 2000 kilo Dalton | |
| 4 | Aloe vera powder | 0.5 |
| 5 | Panax ginseng extract | 1 |
| 6 | Sodium chloride | 0.25 |
| 7 | Potasium phosphate | 0.25 |
| 8 | Sodium phosphate | 0.25 |
| 9 | Magnesium chloride | 0.2 |
| 10 | Calcium chloride | 0.2 |
| 11 | Sodium bicarbonate | 0.2 |
| 12 | Fructose | 0.5 |
| 13 | 1,3 Propendiol | 3 |
| 14 | Germall plus | 0.5 |
| 16 | Purified water | q.s. |

Manufacturing process of gel composition of example 6 is similar to steps mentioned in example 2.

Pharmaceutical composition of present invention is studied for chemical test parameter like glucuronic acid content, hyaluronic acid content, heavy metal content like lead, arsenic, cadmium, mercury, micro-biological parameter like content of E. coli, Salmonella, Staphylococcus aureus, Pseudomonas aeruginosa, yeast and molds. Its results are shown in Table 1.

TABLE 1
Chemical and micro-biological test results

| Chemical test | Acceptable limit | Result |
|---|---|---|
| Glucuronic acid content | Greater than 46% w/w | 46.8% |
| Hyaluronic acid content | Greater than 95% w/w | 98.4% |
| Heavy metal content | Not more than 20 parts | 5 parts per |
| | per million | million |
| Lead | Not more than 3 parts | 1 parts per |
| | per million | million |
| Arsenic | Not more than 2 parts | 0.5 parts per |
| | per million | million |
| Cadmium | Not more than 1 parts | 0.5 parts per |
| | per million | million |
| Mercury | Not more than 0.1 parts | No detectable |
| | per million | amount |
| Micro-biological content | | |
| E. coli | Absent | Absent |
| Salmonella | Absent | Absent |
| Staphylococcus aureus | Absent | Absent |
| Pseudomonas aeruginosa | Absent | Absent |
| Yeast | Not more than 10 cfu/g* | Complies |
| Molds | Not more than 10 cfu/g* | Complies |

*cfu/g = colony forming unit per gram

Pharmaceutical composition of present invention is studied for physical parameter like, appearance, clarity, odour, pH, osmolality, viscosity, irritation test, aesthetic feel. Its results are shown in Table 2.

TABLE 2
Physical test results

| | | Reference | | |
|---|---|---|---|---|
| | | gel 1- | Reference | Negative |
| Physical | | Conceive | gel 2- | Control - |
| test | Test | Plus | pre seed | KY Jelly |
| Appearance | Clear gel | Clear gel | Clear gel | Clear gel |
| Clarity | Transparent | Transparent | Transparent | Transparent |
| | to slight | | | |
| | yellowish | | | |
| Odour | Odourless | slight | slight | Odourless |
| | | odour | odour | |
| pH | 6.8 | 6.5 | 6.6 | 7.5 |
| Osmolarity# | 319 | 350 | 291 | 380 |
| Viscosity$ | 5400 | 6500 | 8000 | 1650 |
| Aesthetic | Gentle | Gentle | Gentle | Gentle |
| feel* | | | | |
| Skin | No | No | No | No |
| irritation* | irritation | irritation | irritation | irritation |
| Texture* | smooth, | smooth, | smooth, | smooth, |
| | non-greasy | non-greasy, | non-greasy, | sticky |
| | | slight sticky | slight sticky | |

#mOsmo/kg
$Centipoise
*test perform on 20 humans by applying gel on hand skin and evaluate through sensory analysis in web chart

Pharmaceutical composition of present invention is studied for long term and accelerated stability condition as per ICH guideline. Its results are shown in Table 3.

TABLE 3
Stability study results

| | | Stability condition | | | |
|---|---|---|---|---|---|
| | | 25° C./ | 25° C./ | 40° C./ | 40° C./ |
| | | 60% RH | 60% RH | 75% RH | 75% RH |
| Test | Initial | 6 Month | 12 Month | 3 Month | 6 Month |
| Appearance | Clear | Clear | Clear | Clear | Clear |
| | colourless | colourless | colourless | colourless | colourless |
| | gel | gel | gel | gel | gel |
| pH | 6.8 | 6.6 | 6.5 | 6.8 | 6.5 |
| Odour | Odourless | Odourless | Odourless | Odourless | Odourless |
| Osmolarity* | 319 | 325 | 322 | 318 | 300 |
| Viscosity# | 5400 | 5320 | 5180 | 5100 | 4995 |
| Phase | Uniform | Uniform | Uniform | Uniform | Uniform |
| separation | Gel | Gel | Gel | Gel | Gel |

*mOsmo/kg
#Centipoise

TABLE 4
Assay during stability period

| | | Stability condition | | | |
|---|---|---|---|---|---|
| | | 25° C./ | 25° C./ | 40° C./ | 40° C./ |
| | | 60% RH | 60% RH | 75% RH | 75% RH |
| Test | Initial | 6 Month | 12 Month | 3 Month | 6 Month |
| Hayeluronic | 99.68% | 98.35% | 98.02% | 97.65% | 97.55% |
| acid assay | | | | | |

Table 1, table 2, table 3 and table 4 shows the composition of present invention complies with chemical parameter, microbiological parameter, physical parameter test and remains stable over the specified period of time.

Pharmacodynamics parameter like Sperm motility, Sperm vitality, Sperm Morphology and Hypo-osmotic swelling test in the human volunteers of the pharmaceutical composition according to present invention is studied and compare with reference gel (i.e. marketed formulation) and negative control. Pharmacodynamics study perform on five individual volunteers. Pharmacodynamics study results are shown in Table 5. Results shown is average of five individual volunteers at each time frame.

TABLE 5
Pharmacodynamics study results

| Experimental | Time frame | | | | |
|---|---|---|---|---|---|
| Groups | 20 Min | 40 Min | 60 Min | 120 Min | 240 Min |

Spermatozoa/Sperm Motility (%) (Initial value - 58%)

| | | | | | |
|---|---|---|---|---|---|
| Test-1 | 54.93 | 47.73 | 46.67 | 39.67 | 24.20 |
| Negative | 3.33 | 0.33 | 0.00 | 0.00 | 0.00 |
| Control | | | | | |
| Reference gel 1 | 43.80 | 38.00 | 27.80 | 21.73 | 12.93 |
| Reference gel 2 | 17.00 | 12.27 | 9.67 | 6.33 | 4.33 |
| Test-1 | 62.13 | 57.78 | 49.73 | 35.27 | 29.27 |
| Negative | 3.80 | 1.06 | 0.00 | 0.00 | 0.00 |
| Control | | | | | |
| Reference gel 1 | 31.40 | 27.11 | 18.73 | 14.07 | 8.87 |
| Reference gel 2 | 13.27 | 11.17 | 8.40 | 5.33 | 2.80 |

Spermatozoa/Sperm Morphology (%) (Initial value - 33%)

| | | | | | |
|---|---|---|---|---|---|
| Test-1 | 30.86666667 | 29.1333 | 26.8 | 22.26667 | 18.33333 |
| Negative | 14.86666667 | 12.9333 | 10.6667 | 9 | 6.6 |
| Control | | | | | |
| Reference gel 1 | 24.66666667 | 20.5333 | 17.8 | 14.26667 | 11.26667 |
| Reference gel 2 | 20.73333333 | 18.6667 | 15.2 | 12.93333 | 11 |

Hypo-osmotic swelling (%) (Initial value - 60%)

| | | | | | |
|---|---|---|---|---|---|
| Test-1 | 64.40 | 61.07 | 55.80 | 45.93 | 39.53 |
| Negative | 5.67 | 2.73 | 0.47 | 0.00 | 0.00 |
| Control | | | | | |
| Reference gel 1 | 37.00 | 32.47 | 26.93 | 20.60 | 13.13 |
| Reference gel 2 | 25.27 | 20.20 | 15.27 | 11.80 | 7.60 |

Here, test 1 or test sample is pharmaceutical composition according to present invention as mentioned in example 6, Reference gel 1 is Conceive Plus, Reference gel 2 is Pre-seed and negative control is KY Jelly.

Pharmacodynamics study results shown in table 5 indicates that gel composition of present invention shows higher percentage of Sperm motility, Sperm vitality, Sperm Morphology and Hypo-osmotic swelling compare to negative control and reference gel (i.e. marketed formulation). Further, results of sperm morphology analysis shows that the morphology of sperm remains same compare to reference gel and negative control.

Hence, gel composition of present invention provides better fertility aid effect to trying to conceive patients compare to already existing marketed product or formulation.

Here, results shown in table 1, table 2, table 3, table 4 and table 5 are pharmaceutical gel composition according to present invention as mentioned in example 6. Further, same results can also be produce within examples of gel, solution and solution for spray. Apart from that results can be reproducing within whole range of invention as mentioned in detailed description and examples.

We claim:

1. A stable topical pharmaceutical composition comprising fertility aid or pharmaceutically acceptable salt thereof and pharmaceutically acceptable excipients.

2. The pharmaceutical composition as claimed in claim 1 wherein fertility aid is selected from the group consisting of sodium hyluronate, L arginine, Vitamins, carbohydrate, electrolytes, extract or mixture thereof.

3. The pharmaceutical composition as claimed in claim 1 wherein fertility aid is selected from the group consisting of sodium hyluronate and panax ginseng extract.

4. The pharmaceutical composition as claimed in claim 2 wherein sodium hyluronate is selected from the group consisting of high molecular weight, medium molecular weight, low molecular weight, oligomer, or mixture thereof.

5. The pharmaceutical composition as claimed in claim 1 wherein pharmaceutically acceptable excipients are selected from the group consisting of gelling agent, soothing agent, preservative, antioxidant, weeting agent, solubilizers, humectant, buffering agent, surfactants, moisturizing agent, emollients, chelating agents, solvents or mixture thereof.

6. The pharmaceutical composition as claimed in claim 1 wherein stable topical pharmaceutical composition is gel, solution or solution for spray.

7. The pharmaceutical composition as claimed in claim 1 wherein stable topical pharmaceutical composition provide at least 85% of Spermatozoa/sperm motility in 20 minute compare to initial Spermatozoa/sperm motility value, at least 55% of Spermatozoa/sperm motility in 120 minute compare to initial Spermatozoa/sperm motility value and at least 35% of Spermatozoa/sperm motility in 240 minute compare to initial Spermatozoa/sperm motility value.

8. The pharmaceutical composition as claimed in claim 1 wherein stable topical pharmaceutical composition provide at least 85% of Spermatozoa/sperm vitality in 20 minute compare to initial Spermatozoa/sperm vitality value, at least 55% of Spermatozoa/sperm vitality in 120 minute compare to initial Spermatozoa/sperm vitality value and at least 35% of Spermatozoa/sperm vitality in 240 minute compare to initial Spermatozoa/sperm vitality value.

9. The pharmaceutical composition as claimed in claim 1 wherein stable topical pharmaceutical composition provide at least 85% of Spermatozoa/sperm Morphology in 20 minute compare to initial Spermatozoa/sperm Morphology value, at least 55% of Spermatozoa/sperm Morphology in 120 minute compare to initial Spermatozoa/sperm Morphology value and at least 35% of Spermatozoa/sperm Morphology in 240 minute compare to initial Spermatozoa/sperm Morphology value.

10. The pharmaceutical composition as claimed in claim 1 wherein stable topical pharmaceutical composition provide at least 85% of Hypo-osmotic swelling in 20 minute compare to initial Hypo-osmotic swelling value, at least 55% of Hypo-osmotic swelling in 120 minute compare to initial Hypo-osmotic swelling value and at least 35% of Hypo-osmotic swelling in 240 minute compare to initial Hypo-osmotic swelling value.

11. The pharmaceutical composition as claimed in claim 1 wherein stable topical pharmaceutical composition is prepared by process consisting of following steps;
a) Transferring required quantity of solvent in a vessel;
b) Optionally, heating the solvent between 60-65° C. with continuous stirring;
c) Adding required quantity of gelling agent, soothing agent, preservative, antioxidant, weeting agent, solubilizers, humectant, buffering agent, surfactants, moisturizing agent and solvents;
d) Stirring the mixture till transparent gel or solution or solution for spray is obtain.
